(19) United States
(12) Patent Application Publication
(10) Pub. No.: US 2005/0228491 A1
(43) Pub. Date: Oct. 13, 2005

(54) Anti-adhesive surface treatments

(76) Inventors: Alan J. Snyder, Hummelstown, PA (US); Christopher A. Siedlecki, Harrisburg, PA (US); Keith Milner, Hershey, PA (US)

Correspondence Address: Douglas L. Wathen;Gifford, Krass, Groh, Sprinkle,, Anderson & Citkowski, P.C., PO Box 7021, Troy, MI, 48007-7021, US

(21) Appl. No.: 11/103,302

(22) Filed: Apr. 11, 2005

Related U.S. Application Data
(60) Provisional application No. 60/561,350, filed on Apr. 12, 2004.

(51) Int. Cl. A61F 2/06; A61F 2/02; B29C 33/40

(52) U.S. Cl. 623/1.46; 623/23.74; 264/227

(57) ABSTRACT

A surface providing reduced adhesion to formed elements, having an element dimension such as formed element diameter, has a plurality of topographic features. The topographic features have a feature dimension less than the dimension of the formed element so as to reduce the accessible area of the surface available to the formed element for adhesion to the surface. The topographic features may include protrusions, such as pillars.

### REFERENCE TO RELATED APPLICATION

This application claims priority to U.S. Provisional Patent Application Ser. No. 60/561,350, filed Apr. 12, 2004, the entire content of which is incorporated herein by reference.

### FIELD OF THE INVENTION

The present invention relates to surfaces providing reduce adhesion of formed elements, such as biological formed elements, to a surface. In particular, the invention relates to methods and apparatus to reduce the adhesion of biological formed elements such as platelets to surfaces in contact with biological fluids such as blood.

### BACKGROUND OF THE INVENTION

Many formed elements, such as cells and platelets, can adhere effectively to smooth surfaces, whether they are flat or gently curved, and may also adhere or reside preferentially in grooves, wells or crevices that are somewhat larger than the formed elements. Such structures may present a large surface area with which the formed element may interact and may further shield the formed element from shear forces due to any fluid motion across the surface. Adhesion of blood cells, bacteria, and the like, to surfaces causes problems in numerous applications.

Presently, materials for contact with biological systems are selected largely for their mechanical and chemical properties. Mechanical properties can provide features such as flexibility, structural integrity and durability appropriate to the application. Chemical properties may be chosen to avoid toxicity, avoid rapid breakdown by substances present in the biological environment, and to minimize unwanted effects such as initiation of inflammatory responses and activation and/or adhesion of formed elements. It is difficult to obtain desired mechanical properties and desired chemical properties in the same material. Furthermore, chemical properties can reduce but generally do not eliminate undesirable interactions between formed elements and synthetic materials.

Synthetic polymers are a commonly used class of materials for blood-contacting medical devices. An important group of materials within this family are the polyurethanes. While polyurethane biomaterials have shown some level of success, these materials often require the recipient to receive pharmaceutical anticoagulation therapy and to be exposed to the concomitant risks of such therapy. This therapy is necessary at least in part due to the risk of thromboembolic events secondary to the formation of surface-induced thrombi. Generally, chemical approaches have been used to alter the adsorption of proteins as well as the adhesion of platelets and cells to materials, by either changing the base material or by selectively modifying the physical and chemical surface properties.

The successful design of blood-contacting biomaterials suitable for long-term implantation remains a significant challenge to the treatment of cardiovascular disease with implantable medical devices. While still not ideal, polyurethane biomaterials have shown suitable mechanical properties and acceptable biocompatibility for many applications. Polyurethane materials have been used as heart valve materials, vascular grafts and as the flexible blood-contacting components of circulatory support devices.

Previous topographic strategies in blood-contacting biomaterials may be summarized by two contrasting approaches: the application of large scale (many microns) textured materials to encourage the formation of an adherent neointima consisting of biological materials such as fibrin, cells and cell fragments; and the combination of very smooth surfaces and carefully selected pump geometry to enable efficient washing and discourage platelet-surface adhesion. It is clear that the typical response to supra-cellular features that provide large surface areas and possibly disturb blood flow is enhanced adhesion and cell spreading.

In blood contacting devices, it is common to choose a material for its relative resistance to formed element adhesion or activation, and this relative resistance is augmented by arranging for sufficiently vigorous blood flow to wash the surface through fluid shear stresses. In applications such as food handling, materials may be fabricated to be free of crevices that are hard to clean or provide preferential spaces for bacterial adhesion and growth, and this is augmented by periodic cleaning with detergents or antiseptics.

Previous work using materials fabricated with supracellular ordered textures typically attempts to promote cell adhesion and control the direction of cell growth. Random micro- or nano-scale texturing processes have the same aim: inducing an increase in cellular adhesion, often for tissue engineering applications.

### SUMMARY OF THE INVENTION

A surface provides reduced adhesion to formed elements, the surface having topographic features having a feature dimension less than an appropriate dimension of the formed element. The feature dimension can be the spacing between surface protrusions, such as pillars, ridges, or other protrusions, or the width of a channel or other indentation (such as channel width, pit width, or pit diameter for a circular pit), or other dimension of related to topographic features. The term protrusion refers to, for example, a topographic feature extending away from the bulk of the material providing the surface. The appropriate dimension of the formed element may be a diameter, approximate diameter, average diameter, width, or other dimension relevant to interactions between the formed element and the surface.

For reduced adhesion of platelets and similarly sized formed elements, topographic features may be pillars, for example pillars each having a pillar width and a pillar height of between approximately 100 nm and 1000 nm, and a pillar spacing between 300 nanometers and 1000 nm. Feature dimensions can be correlated with formed element size.

A surface according to an example of the present invention can form part of a blood pump or vascular graft, and can be used in medical implants. A surface according to an example of the present invention can provide reduced adhesion to bacteria, and used in any application where bacteria are a problem, such as a food preparation surface.

A reduced adhesion surface can be formed in any appropriate material. For blood pump and vascular graft applications, a synthetic polymer such as a polyurethane can be used. An improved vascular graft comprises a polymer tube, the interior surface of which has topographic features having a feature dimension less than the effective diameter of a platelet.

A process for fabricating a surface having reducing adhesion of formed elements includes providing topographic features having a topographic dimension less than an approximate dimension (such as an approximate diameter) of the formed element. For example, pillars or ridges can be provided, having a pillar or ridge spacing less than the diameter of a blood component such as blood cells or platelets.

### BRIEF DESCRIPTION OF THE DRAWINGS

FIGS. 1A and 1B are schematic illustrations showing platelet interaction with textured and smooth biomaterial surfaces;

FIGS. 2A-2F show a fabrication protocol for preparation of synthetic polymer replicates of nanofabricated silicon wafers;

FIG. 3 is a flow chart of a fabrication protocol for preparation of surfaces in synthetic polymer;

FIG. 4 shows a SEM image showing an array of pillars having a pillar width of 300 nm pillars and a pillar spacing of 300 nm, fabricated in poly(urethane urea), PUU;

FIG. 5 shows a platelet on a surface according to an example of the present invention;

FIG. 6 shows a SEM image showing an array of pillars having a pillar width of 700 nm pillars and a pillar spacing of 700 nm fabricated in PUU;

FIGS. 7A and 7B show SEM images of platelets adherent to a 700 nm/700 nm (pillar width/pillar height) textured material near the center (zero shear) and at 51 dynes/cm² after a rotating disk experiment; and

FIGS. 8A and 8B shows adhesion coefficients for smooth and nanotextured PUU materials.

### DETAILED DESCRIPTION OF THE INVENTION

Adhesion of formed elements to a surface is dependent on the formed elements accessing the material surface. Biological formed elements such as platelets and bacteria may access the surface through mediating adhesion molecules and/or molecules which coat the synthetic surface. An example surface having reduced adhesion to formed elements comprises topographic features that reduce the surface area available for adhesion. The topographic features may include protrusions, extending generally away from the bulk of the material, such as ridges, pillars, and the like. The topographic features may also comprise indentations, such as pits, troughs, and the like.

Adhesion of biological formed elements such as cells, platelets, and the like to surfaces can cause problems in many situations. For example, adhesion of platelets to surfaces may lead to thrombosis, as discussed in more detail below. Examples discussed in this specification sometimes are directed towards platelets as the formed element of interest. However, these examples are not intended to be limiting. Examples of the present invention can be used to reduce adhesion of other biological and non-biological formed elements.

A surface in a synthetic material suitable for implantation in a living subject, or otherwise in contact with a biological system, comprises topographic features having a feature dimension less than a dimension of the formed element. The topographic features can significantly reduce the surface area available for interaction between the surface and the formed elements.

The present invention can be used with a broad range of materials, such as those that encounter a biological environment whether by implantation, extracorporeal processing of blood or other body fluids, or materials used in food and beverage handling and work surfaces that encounter biological formed elements such as bacteria. The term polymer includes synthetic polymers, polymeric biomaterials, biopolymers, and other polymers. The term biomaterial includes all materials that may contact cells or tissues, including polymers, and non-polymers such as metals and inorganic materials. The surface patterns described herein may be formed on a variety of synthetic and natural materials intended for uses such as those just described, and the principle of deprivation of formed element access to biomaterial surface can be the same regardless of the composition of the biomaterial.

The effective surface area to which platelets contact can be reduced to a small portion of the nominal platelet footprint while still keeping the largest continuously available area below of the platelet diameter. The lithographic masks were drawn with square pillars, as they are simpler to draw in the electron beam machine used to make the masks, but at nanoscale size the pillars often are formed with a circular cross-section in the UV lithography process because they are only a few wavelengths across. As an example of reduction in contact area without access to smooth areas between features, consider a rectangular array of circular pillars of radius R and are spaced on 4R centers, i.e. there is a distance 4R between pillar centers and a pillar spacing of 2R along a side of the rectangular array. The proportion of area covered by pillars is (πR²)/(16R²), or 19.6%. Hence, the available surface area of the nanotextured surface is <20% of a smooth surface. To ensure that the formed element cannot fit between the pillars, the pillar spacing can be less than the formed element diameter. For pillars on 4R centers in the X or Y direction, they are on (4R.√2) centers diagonally. The space between them is (4R.√2)−2R, or 3.66R. For 700/700 dimension pillars (nm diameter/spacing), 3.66R is 1.28 microns, which is about the diameter of a platelet. For 400/400 pillars, 3.66R is equal to 0.73 microns, or about 50% of the platelet diameter.

In examples of the present invention, a surface has a plurality of pillars, and the largest separation distance of the pillars is less than the formed element dimension. If the formed element uses adhesion proteins in interaction with the surface, the pillar height can exceed adhesion protein reach, and prevent the formed element from reaching the base by deformation or its geometry. The area covered by pillars can be made as small as practically possible, as long as the pillars are not so thin that they fall over.

Hence, for a given pillar height, the pillars have a minimum diameter in order to be sufficiently stiff that they don't fall over and provide extra accessible surface. To minimize the total accessible surface area, the pillars can be as far apart as possible but not so far apart that there is any space large enough for the formed element to fall into. For rectangular (including square) arrangements, a diagonal pillar spacing is likely to be significant. However, pillars need not be on a rectangular array pattern; they can also be arranged in staggered rows, for example to form an array of equilateral triangles.

A surface may have topographic features having different size ranges, for example to modify adhesion properties of two or more species of formed element. A surface may also include areas each characterized by having different topographic features or feature sizes, for example to provide a surface having a different adhesion properties in different areas.

In examples of the present invention, a pillar may have a square, circular, or other shaped cross-section. The pillar cross-section may be substantially uniform along most or substantially all of the height of the pillar, or may taper as the pillar extends away from the rest of the surface. The pillar side walls may be generally orthogonal to the surrounding surface. A pillar may have a generally flat or a rounded cap. For example, a pillar may be generally cylindriform, with a flat or domed top. The pillar has lateral and longitudinal widths, in orthogonal directions within the general plane of the surface. In representative examples of the present invention, both lateral and longitudinal widths are less than the formed element dimension. In the example of a generally cylindriform pillar having a circular cross-section, the lateral and longitudinal widths are the same. Topographic features may include pillars having different feature dimensions.

Examples of the present invention also include surfaces having ridges, which may have a lateral width (orthogonal to the ridge) and lateral spacing that are both less than the formed element dimension, and a longitudinal width (along the ridge) that is substantially greater. However, ridges need not be straight, they can also be formed in curves, whorls, concentric rings, geometric patterns, or other configuration.

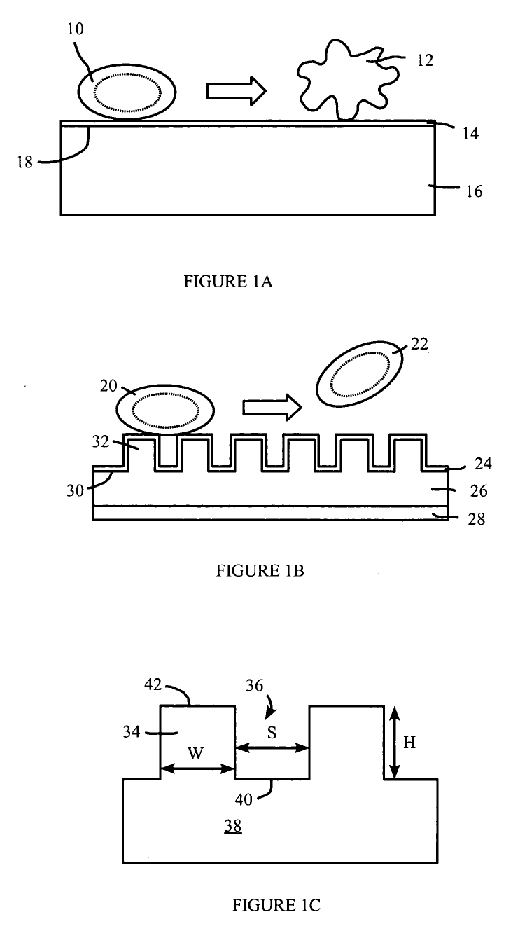

FIG. 1A is a schematic illustrating a typical interaction between a platelet 10 and the surface 18 of a synthetic polymer 16, which may for example be a part of an implanted medical device. When exposed to blood, the polymer surface 18 becomes coated with a thin protein coating 14. The platelet 10 adheres to the surface, and is activated, represented by the irregular shape 12. The activated platelet releases further proteins, which tend to stimulate formation of an aggregation of activated platelets and to stimulate polymerization of fibrinogen via the clotting cascade. If the aggregated platelets and/or fibrin break free of the surface, and the resultant thromboembolism may cause serious injury such as stroke.

FIG. 1B illustrates interaction between a platelet 20 and a surface 30 according to an example of the present invention, the surface 30 being formed in a synthetic polymer film 26 and illustrated here in cross-section. As discussed above, the surface 30 becomes coated with a thin protein coating 24 on exposure to blood. The topographic features of the surface 30 include pillars or ridges 32, having a width, height, and spacing. The polymer film 26 may optionally be formed on a substrate 28. The substrate and polymer film may both be PUU, poly(urethane urea), or the polymer film may be any biocompatible polymer, and the substrate may be any suitable substrate material.

In applications where a protein coating or analogous conformal coating by material from the bulk fluid forms on the surface, the topographic feature size are preferably large enough that the protein coating does not substantially smooth the surface, for example by filling in gaps between the pillars. Also, the spacing between the pillars may be slightly greater than the diameter of the formed elements, if subsequent surface film formation reduces the spacing to less than or approximately equal to the diameter of the formed element.

FIG. 1C further illustrates a portion of a surface formed on material 38, the surface having topographic features such as protrusion 34, such as a pillar having width W and height H, and a spacing S from an adjacent pillar.

An example surface according to the present invention has a topographic feature size that lowers the area accessible by the formed element. The pillar spacing S can be less than the diameter of the formed element to prevent the formed element accessing the base of the groove or pillar 42. Only the upper surface 44 of the pillars provides accessible area for adhesion of the formed elements.

Several types of biological formed element adhere to a surface using adhesion proteins, and in such cases preferably the pillar height H is greater than the range of the adhesion protein. For any formed element, according to the mechanical properties of the material 40, spacing S, height H and width W can be chosen so that neither the sides of the protruding features nor the base are accessible to the formed element either by excessively large spacing or by deformation of the protruding features or deformation of the formed element.

For patterns formed from pillars, the cross-sectional shapes of the pillars may be chosen according to convenience in manufacture or to best suit the material and the formed element. For example, circular pillars may be most readily manufactured using certain lithography equipment or if formed directly upon a laser-drilled master, while other cross-sections including for example “X” or “C” shapes that resist bending may be readily formed as well and permit further reduction in available surface area without fear of exposure of pillar sides to the formed elements. Likewise, patterns comprising ridges and grooves may be formed in wavy patterns in order to best resist bending in the presence of flow and/or formed element contracture forces. Such ridges and grooves may be continuous across the extent of the material or may be interrupted. In experiments to date, patterns were formed across large areas using stepper lithography. Using this method, best efforts at registration of successive patches resulted in some variation in pillar spacing at the boundaries between patches, yet greatly reduced adhesion overall was obtained.

For a rectangular array of pillars having a spacing S along the rows (or columns) of pillars, the largest areas between pillars will be determined in part by the pillar spacing along a diagonal, which can be more than S. Alternative pillar shapes and distribution patterns may be readily derived that best deprive formed elements of access to the base while keeping the accessible areas at the tops of the protruding features low.

In another example, the protrusion 34 is a ridge having width W and height H, and spacing S from an adjacent ridge. Equivalently, in this example, the surface may be seen as having a groove 36 having width S and depth H, the grooves having a spacing W. If the topographic feature is a groove, the width of the groove can be less than the diameter of the formed element.

In examples of the present invention textured surfaces having ordered square arrays of circular pillars were fabricated. In some samples, pillar widths and pillar spacings of approximately 700 nm were used. In others, pillar widths and pillar spacings of approximately 400 nm were used. In all samples, pillar heights were approximately 680 nm. The protein coating appeared to be less than 100 nm thick. Platelet diameters are typically 1.0 to 1.5 micron before activation and approximately 3 micron after activation.

A formed element interacting with a surface such as represented by FIG. 1B has less average contact area between the formed element and the surface. In an example where the formed element is a platelet, there is a reduced contact area between membrane receptors of the platelet and the adsorbed protein coating, compared to the smooth surface shown in FIG. 1B where adhesion proteins have access to the entire surface area beneath the cell. Hence, the platelet has a reduced ability to maintain contact in the presence of fluid shear, and consequently has a reduced ability to activate and spread. Only portions of the pillars are available for interaction with the surface if the heights of the pillars are larger than the distance over which the cellular adhesion proteins can reach. Preferably, the pillars have height and spacing such that a formed element, such as a cell, cannot deform sufficiently for adhesion to occur between pillars. The formed element may partially adhere to the surface but the more tenuous attachment renders the formed element more readily removed by fluid flowing past.

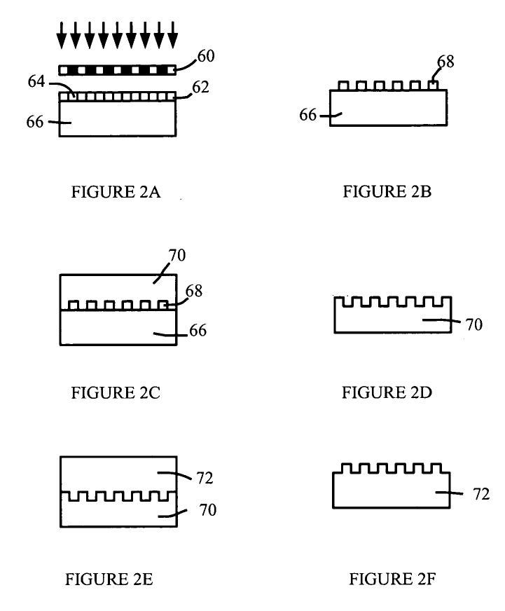

FIGS. 2A-2F illustrate a fabrication process for preparation of synthetic polymer surfaces according to examples of the present invention. In this example, poly(urethane urea) (PUU) replicates of nanofabricated silicon wafers are formed, but the surface may be formed in other polymers, other materials, or using different mask materials, lithography techniques, and other process variations.

FIG. 2A corresponds to using a mask 60 to expose a photoresist layer 62 on a silicon wafer 66. Portions of the photoresist, such as inside region 64, are shaded from irradiation by the mask 60. FIG. 2B represents the structure obtained after exposure and development of the resist layer, in which topographic features are provided by photoresist remaining after development. FIG. 2C represents creating a silicone negative 70 by casting uncured silicone elastomer (silicone rubber) over the wafer 66 and allowing the silicone to cure. FIG. 2D corresponds to removing the silicone negative 70 from the wafer. FIG. 2E represents casting a polymer film 72 on the silicone negative 70, which is used as mold upon which the polymer film 72 is cast. FIG. 2F corresponds to removing the polymer film 72 from the silicone negative, the polymer film having a surface topography that duplicates that formed on the silicon wafer 66 as shown in FIG. 2B.

The term ‘polyurethane’ includes block copolymer materials that generally have either an ether or ester soft segment (in biomedical polyurethanes) with an aromatic or aliphatic hard segment and a urethane or urethane urea linkage. The term polyurethane, as used herein, includes such poly(urethane urea) polymers having a urethane urea linkage. However, surfaces according to the present material can be formed in any material, such as other biocompatible materials for implant purposes.

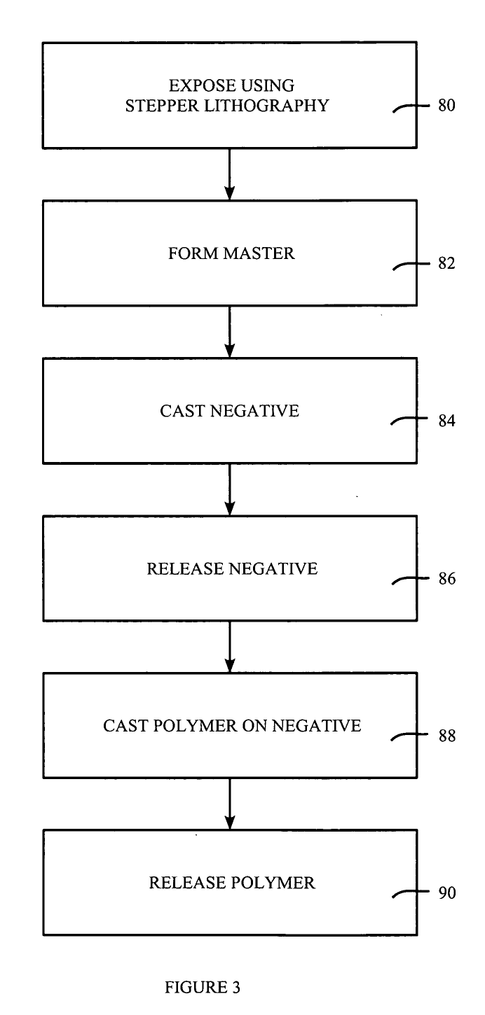

FIG. 3 is a flow chart representing formation of topographic features in a surface, along with a summary of an example process. Box 80 represents exposing a photoresist layer on a substrate using a lithography process, such as stepper lithography. Box 82 represents formation of a master after the lithography process. Box 84 represents casting of a silicone rubber negative. Box 86 represents releasing the silicone rubber negative. Box 88 represents casting the polymer layer on the silicone rubber negative, the surface being formed on the polymer layer, the surface duplicating that formed by the photoresist layer on the substrate. Box 90 represents releasing the polymer film from the negative. Further process details are discussed further below. This two-stage replication molding process provides a simple an economical means of forming patterns in a material such as polyurethane, whose carrier solvent would dissolve photoresist. Other lithographic and casting processes may be used. For example, a negative master may be formed by photolithography followed by etching of underlying silicon, or directly by electron beam or laser drilling.

FIG. 4 shows a SEM image showing an array of 300 nm pillars fabricated in PUU material. The 300 nm pillar size was the limit of the optical lithography step used to fabricate the master and represented the smallest features that were fabricated in PUU. If higher resolution lithography such as electron beam lithography is used, smaller feature sizes and details in cross-sectional shapes may be formed. FIG. 4 shows a 5×5 array of pillars 300 nm in diameter and spaced 400 nm apart. Atomic force microscopy indicated a 640 nm pillar height, similar to the nominal 700 nm thickness of the original photoresist.

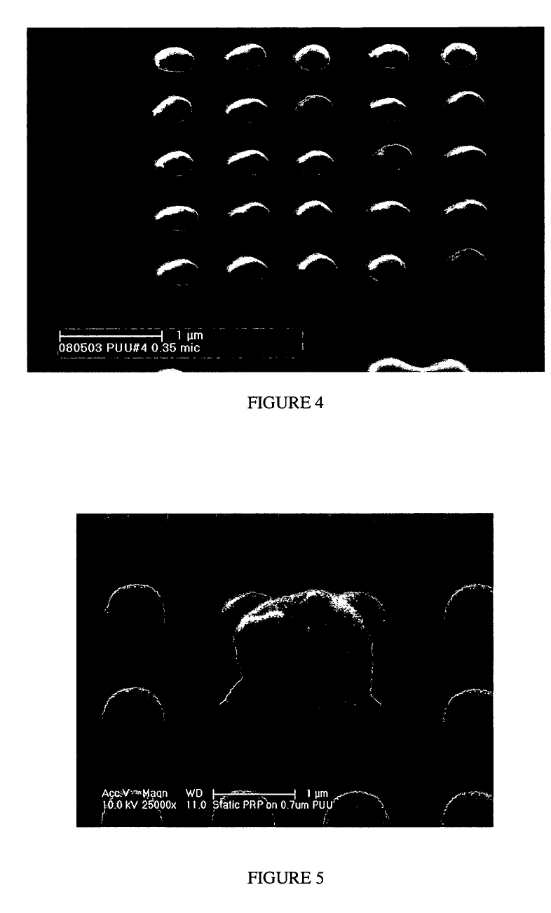

FIG. 5 shows a platelet on a PUU surface having 700 nm pillar widths and 700 nm pillar spacing. The contact area between the platelet and the PUU surface is reduced by the topographic features.

FIG. 6 is a scanning electron microscopy (SEM) image showing an array of 700 nm pillars, the pillars separated by a spacing of 700 nm. Sampling multiple substrates indicated that 99.7% of pillars are properly replicated. For SEM imaging, the polymer (PUU) samples were coated with 10 nm of gold and imaged in a high voltage Philips XL-20 SEM. The topographic features imaged included a series of lines and pillar arrays of different sizes ranging from 300 nm to 750 nm (pillar width and/or spacing).

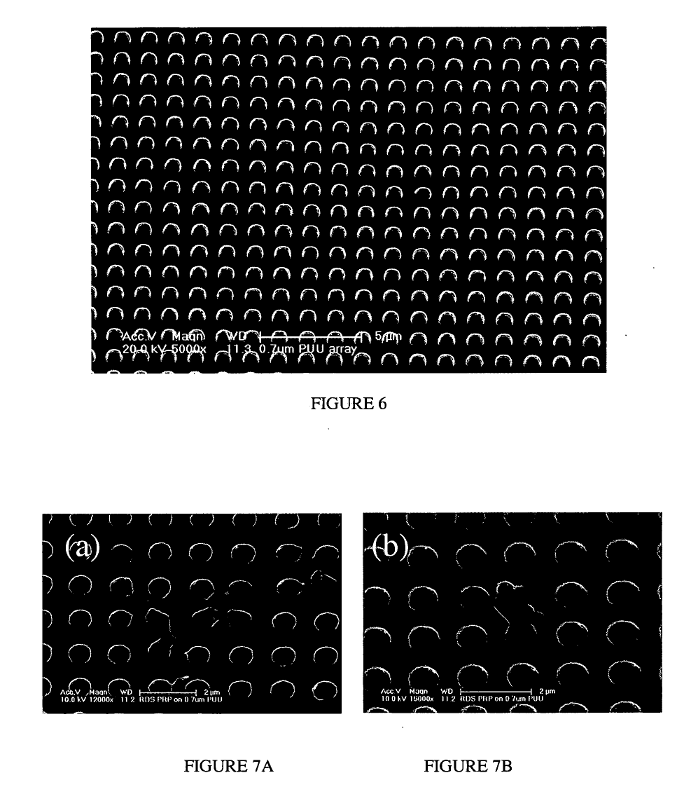

FIGS. 7A and 7B show SEM images of platelets adherent to a 700 nm pillars spaced/700 nm apart (a) near the center (zero shear) and (b) at a 51 dynes/cm² region of the substrate after a rotating disk experiment. The few platelets seen on this material were usually seen in the areas between the textured pillars, consistent with the understanding described above that fabrication of pillars with smaller spacings will be even more efficient in reducing platelet adhesion.

The adsorbed protein coating does not appear sufficiently thick to substantially modify the textures at this feature size, verifying the assumption that the protein layer is thin and conformal to the textures.

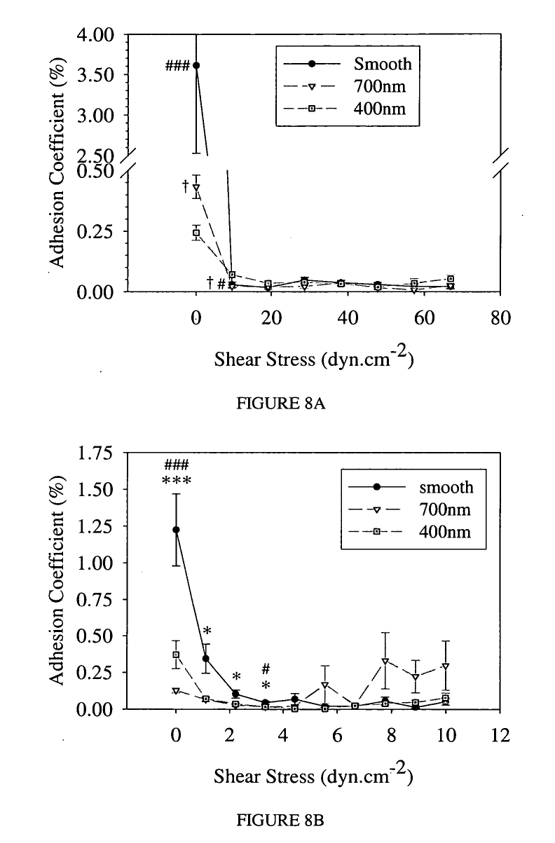

FIGS. 8A and 8B show adhesion coefficient results for surfaces having 700 nm/700 nm and 400 nm/400 nm posts (pillar width/spacing), compared with a smooth PUU film cast on the same Sylgard 184 silicone. The adhesion coefficients versus shear rates were taken using a spinning disk sample in platelet rich plasma. Statistical significance is denoted using the symbol (*) when comparing smooth and 700 nm PUU, the symbol (#) comparing smooth and 400 nm PUU, and the symbol (554 ) comparing 700 nm-400 nm PUU. One symbol denotes P<0.05, two symbols denote P<0.01 and three symbols denote P<0.001.

In the experiment corresponding to FIG. 8A, the spinning disk had approximately 100 micrometer lateral wobble in its rotation, which provides a slight non-zero flow at the disk center and possibly a small disturbance of the boundary layer in all samples. Although adhesion coefficients were low for both films, adhesion coefficients were found to be significantly lower (p<0.05) for most of the shear stresses studied. The error bars indicate standard error of the mean. Stars indicate statistically significant differences in the adhesion coefficients. These results may correspond to actual applications where transient flow disturbances are normal, such as pump and valve applications, and also in conduit applications due to changing flow.

FIG. 8B shows adhesion coefficient results for surfaces having 700 nm/700 nm and 400 nm/400 nm posts (width/spacing) compared with a smooth PUU film cast on the same Sylgard 184 silicone using the methodology just described for FIG. 8A, except that all wobble was removed from the spinning disk system by careful machining of the sample holder. Six samples at each post size and spacing, and four smooth samples were used in this experiment. The elevated adhesion coefficients for some shear values for the 700 nm/700 nm pattern were due to aggregates that were observed in two of the six 700 nm/700 nm samples. These data clearly demonstrate that texturing of the PUU films has the potential to result in lower platelet adhesion compared to smooth PUU films, particularly at low wall shear stress values that normally present device designers with the greatest concern with respect to formed element adhesion. Further details of adhesion coefficient measurements are described below.

Fabrication of Surface Topographic Features

Replication of photoresist patterns in poly(urethane urea) (PUU) used an intermediate silicone rubber (poly(dimethlysiloxane), PDMS) negative in a replica molding process. PUU cannot be cast directly over patterned photoresist because its carrier solvent would remove the photoresist. Furthermore, PMDS molds may be reused many times with minimal loss of pattern resolution, whereas the UV-5 photoresist may be damaged during the peeling process.

Negative replicas of the master pattern were created by casting Sylgard 184 silicone elastomer on the silicon-photoresist master. Positive PUU replicates were obtained by pouring or spin casting solvent-borne PUU on the silicone negative.

Patterns with the desired topographical arrangements can be fabricated on photoresist-covered silicon wafers using standard procedures. An example process is described below.

1. A hexamethyldisilazane (HMDS) adhesive primer can be applied to a 150 mm diameter silicon wafer using a spin coating system, rotating at 2500 rpm for 5 sec, followed by Shipley UV-5 DUV positive photoresist, applied at 1000 rpm for 12 sec and then spun at 2350 rpm for 40 sec for a thickness of 0.7 micron. Alternatively, an antireflective coating may be used instead of the adhesive primer to limit reflections during the exposure process, thereby improving the quality of the features formed in the photoresist.

2. The photoresist can then be soft baked at 130° C. for 60 sec.

3. The desired pattern can be drawn using layout editing software such as the L Edit Layout Package (Tanner EDA), followed by manufacture of a reticule containing this pattern.

4. The positive photoresist may then be patterned by exposure, for example with a 248 nm excimer laser through the reticule using suitable photolithography optics. As an example, a Nikon NSR Series Stepper provides replication of the pattern over a large area. A suitable exposure does for UV-5 DUV positive photoresists is 12 mJ/cm².

5. The photoresist can then undergo a post-exposure bake at 135° C. for 90 seconds. The photoresist pattern can then be formed by chemical development, for example in Shipley Microposit MF CD-26 for 40 sec. This may be followed by a final hard bake at 145° C. for 3 min. The regions of photoresist exposed to the excimer laser can have been released from the silicon wafer and the regions not exposed can remain on the silicon. This simple process can be used to create photoresist features on the silicon wafer with heights equal to the photoresist thickness, 700 nm or slightly less using the photoresist and dispensing processed described above, and widths and lengths defined by the L-Edit layout. The minimum feature size possible with the Nikon NSR Stepper is approximately 400 nm, but circular features of somewhat smaller diameter may be formed as well.

A silicone negative of the photoresist master pattern can then be formed using a material such as Sylgard 184 silicone elastomer (Dow Corning), for example using the following process.

1. A two part silicone material can be mixed at a ratio of 10:1 base:curing agent (w/w), followed by degassing in a dessicator under vacuum in order to remove any bubbles formed during the mixing. To assist in bubble removal, the vacuum can be released and reapplied several times during this step.

2. The silicone material can be poured over the photoresist master pattern, vacuum degassed further to improve the conformity of the silicone to the features in the photoresist pattern and then cured, for example at 65° C. in a vacuum oven for 4 hrs.

3. The silicone negative can then be peeled from the master. This can easily accomplished by hand. The ratio of base to curing agent and the curing temperature and duration may be altered to improve the replication of the photoresist features in the silicone negative. Replication of 300 nm diameter posts has been demonstrated. Smaller features can be replicated as long as the features are significantly larger than the monomer from which the casting polymer is made and conformity to the master is achieved. Addition of a viscosity-reducing agent such as Dow Corning 200 Fluid 20 cS may be useful for improving conformity of the polymer.

4. The silicone negative may be rinsed in acetone in order to remove any photoresist transferred to the silicone. Alternatively, acetone may be applied to the silicone negative using a spin casting apparatus.

A replica of the photoresist master pattern can be fabricated in a polymeric biomaterial such as a segmented polyether(urethane urea) (PUU). An example PUU is Biospan MS.4 (The Polymer Technology Group, Berkeley, Calif.) supplied as 22.5% solids in dimethyl acetamide (DMAC), having a methylenediisocyanate hard segment, a polytetramethlyene oxide soft segment and an ethylene diamine chain extender. Biospan MS.4 is provided with polymer chains end-capped with 2000 molecular weight poly(dimethlysiloxane) at 0.4% by weight. This material is known to show good durability, little degradation and good compatibility during a variety of animal experiments. Below is an example process which was used to form the surface in PUU. This process can be modified as desired.

1. The PUU may be poured onto the center of the silicone negative, which has been premounted on the chuck of a spin coater such as Model P6700 (Specialty Coating Systems Inc). Alternatively, PUU may be cast by dipping or pouring.

2. For spin casting, spin speed is generally chosen to obtain a uniform coating of PUU on the silicone negative. Typical spin speeds can range from 300 to 1000 rpm, in order to obtain a thickness less than 100 microns. Film thickness can be measured by a number of means such as magnetic ball probes, ultrasound, micrometer measurements and the like, and can be correlated with spin speed and spin duration. The concentration of the base material in its solvent can also be adjusted for different cast thicknesses. In the case of dipping, thickness may be controlled by varying the number of dips and the speed of insertion and removal. Thickness may be selected for the particular application, in order, for example, to obtain desired durability or optical properties.

3. The PUU film can then be degassed under vacuum, for example with the vacuum being broken several times, to remove bubbles and improve conformity of the PUU to the silicone.

4. The PUU can then be cured at room temperature, for example in a vacuum oven for 24 hours and then at 60 degrees C. in a vacuum oven for an additional 24 hours.

5. The silicone/PUU can then allowed to cool, and then for example immersed in deionized water for 60 min to facilitate removal of the PUU replica from the silicone negative.

In order to demonstrate differences in formed element interactions with the material due to the presence of the patterns, patterned PUU surfaces and smooth PUU controls were prepared by spin casting PUU onto clean glass coverslips, matching the thickness of the nanofabricated PUU films as determined by a magnetic ball micrometer. Samples can then be degassed as described in step 4 above, followed by curing for 24 hrs at room temperature and 24 hrs at 60 degrees C. in the vacuum oven, again matching the process used for the nanofabricated PUU films. These test materials can have no exposure to PDMS, and can serve as surface chemistry, fibrinogen adsorption, and platelet adhesion controls for the PDMS replicated PUU materials. In order to determine the effects of low molecular weight PDMS oligomers, if any, that may be transferred to the PUU material during the replication molding process, smooth controls may be prepared by dip casting or spin casting over smooth PDMS.

To test whether the molding process affected the surface chemistry of finished materials, a series of PUU samples were prepared using either the replication molding procedure or cast in a glass dish. The samples were analyzed using a Kratos Analytical Axis Ultra XPS at 90 degree takeoff angle. The approximate sampling depth under these conditions is 80 Angstroms. The surface elemental composition measured by XPS are shown in Table 1 below (hydrogen cannot be detected by XPS).

TABLE 1

| | | | | |
|---|---|---|---|---|
| Sample | C(%) | O(%) | N(%) | Si(%) |

| | | | | |
|---|---|---|---|---|
| glass-cast-air-side | 64 | 21 | 1.4 | 13 |
| glass-cast-surface-side | 66 | 21 | 1.5 | 12 |
| PDMS-cast-air-side | 66 | 21 | 1.6 | 11 |
| PDMS-cast-surface-side | 64 | 22 | 1.7 | 12 |

Although the silicon content is higher than expected for a typical PUU, the consistency in silicon content suggests that poly(dimethylsiloxane) (PDMS) is not being transferred during the replication molding process. Rather, the silicon observed on the sample reflects the preferential accumulation of this PUU's silicone end caps at the air interface.

Any microfabrication techniques can be used to make a master of the topography pattern on a substrate such as a silicon wafer. Masters may be fabricated for example by patterning a photosensitive polymer on the silicon surface, by etching the pattern in to the silicon wafer or by processes such as electron beam lithography in other materials. Use of ultraviolet sensitive photoresist without etching of silicon is cost-effective, especially at low quantities but details of the topographic features that may be formed are limited by the optical process. Etching of silicon or lithography in more durable polymers such as poly(methylmethacrylate) can provide more durable masters. Processes such as electron beam lithography can provide the ability to form more precise features. The substrate may alternatively be a metal, plastic, other inorganic material, or other material.

A 150 mm wafer was coated with 700 nm thick photoresist and patterned with Nikon test reticule, i.e. a Nikon test pattern on a Nikon NSR Series Stepper. The size of the wafer makes it suitable for fabrication of blood pumping diaphragms of having surfaces according to examples of the present invention.

A number of alternative replica molding processes exist. For example, a negative pillar pattern may be etched in silicon or polymethyl-methacrylate and polyurethane may be cast directly upon this negative. Coatings may be used to improve wetting or reduce adhesion at any step. According to the application, use of positive or negative masters fabricated using available methods with direct casting of the polymeric biomaterial or use of one or more intermediate replicates may be found to be most reliable and economical.

Pillar sizes and spacings ranging from 400 nm to 1600 nm (1.6 microns) and larger are easily achieved using optical techniques. X-ray lithography, electron beam (e-beam) lithography, and other techniques can be used to obtain smaller features. Alternatives to the optical lithography described above include laser ablation, scribing, techniques based on scanning microscopy, and the like.

Adhesion Coefficients

Platelet adhesion under steady-state fluid flow conditions was studied using an RDS (rotating disk system). The RDS provides a well-defined and reproducible dynamic flow environment. The shear stress in such systems has been derived under certain simplifying assumptions, namely that the disk is of infinite size and rotating in an infinite medium, that laminar flow exists in the boundary layer at the material surface and that steady-state conditions apply. Under suitable conditions all three of these assumptions are met for practical purposes and the surface shear stress, τ_{S} (dynes/cm²), in the boundary layer at a radial distance x (cm) is given by

$${τ}_{S} = 0.8 η x \sqrt{\frac{{ω}^{3}}{v}}$$

where η is the medium viscosity (poise=dyn sec/cm²), which is 0.011 poise for plasma.

The first of these conditions is met when the edge effects on the disk are negligible, which occurs when the disk radius and the separation between the disk and medium container are both much larger than the boundary layer thickness. The second condition is met if the Reynolds number of the system does not exceed 105. The third condition is fulfilled by operating the apparatus at a steady rotational speed. One may use instruments such as a Pine AFMSRX Analytical Rotator (Pine Instrument Company, Grove City Pa.). This allows acceleration to full speeds up to 1000 rpm in 4 msec and then maintenance of rotational speed within 1% indefinitely. Steady-state conditions occur quickly once stable angular velocity is achieved.

Experiments were performed with an angular velocity of 104.7 rad/sec or 1000 rpm, developing a shear stress at 0.7 cm radial distance of 60.3 dyn/cm². Under this condition the boundary layer thickness is calculated to be 385 microns, which is considerably smaller than the 7500 micron radius of the rotating disk. In addition, the dimensions of the platelets (˜1 micron) and the patterned features on the polyurethane (<700 nm) are considerably smaller than the boundary layer ensuring laminar flow in this region. The relative dimensions of the 50 ml PTFE beaker and the 15 mm polyurethane sample are also considerably larger than the boundary layer thickness, satisfying the conditions for an infinite system. The angular velocity of 104.7 rad/sec gives a system Reynolds number of 4909. Laminar flow therefore exists in the boundary layer.

The adhesion coefficient, AC, may be calculated for each region of the PUU disk where platelet counts are performed. The AC is defined as the ratio of the number of platelets adhered to an area of PUU (N=platelets/mm²) to the number of cells transported to the PUU surface during the experiment. This is expressed as a percentage using

$$AC ( \% ) = 100 \frac{N}{jt}$$

The number of cells transported to the PUU surface is given by the product of the cell flux, j (cells/sec/mm²), and the experiment duration, t (sec). In essence, AC measures the efficiency with which platelets attach to the PUU sample and can equal 100% when all platelets adhere. The cell flux may be calculated from the diffusivity of platelets in PRP, the bulk platelet concentration in the PRP and the reaction rate coefficient at the PUU surface. Assuming a platelet radius of 1×10⁻³ mm, a platelet concentration of 2.5×10⁸ platelets/ml (250 k/microliter) and a temperature of 25 degrees C. leads to a cell flux of 52.3 platelets/mm²/sec.

Platelet-surface interactions may be involved in the formation of emboli without the platelets having first adhered to the surface, because platelet-surface interactions may cause platelet activation without platelet adhesion. To test for platelet activation, platelets that were exposed to the RDS system but were nonadherent were labeled for flow cytometry using a dual labeling technique. A FITC label was used to detect CD41 and to classify objects as platelets, while a PE CD62P label was used to classify platelets as activated. Samples were exposed to either a 700 nm/700 nm pillar/spacing PUU material or a smooth PUU material, both having been prepared by replication molding. These samples were compared to platelets that were allowed to sit undisturbed in the water bath during the RDS experiment. Results are summarized in Table 2, the mean being for a minimum of 3 samples.

TABLE 2

| | Mean Activation |
|---|---|
| Substrate | Percentage (%)* |
| Static Control | 2.1 ± 0.4 |
| Smooth PUU | 4.0 ± 1.9 |
| 700 nm/700 nm | 2.1 ± 0.5 |
| Textured | |

The data in Table 2 indicate that exposure to the 700/700 texture is similar to the static control, while exposure to the smooth material results in higher platelet activation than does the static control.

The morphology of adhered platelets is an indicator of their level of activation. Unactivated platelets tend to be circular and their area is close to P²/4π where P is the perimeter, while activated platelets that have spread tend to include pseudopods and the perimeter is much larger for a given area. The circularity index, P²/4πA where A is the measured area, provides a measure of activation for adhered platelets. The circularity indices for platelets adhered to both textured (700 nm/700 nm) and smooth PUU materials were measured and these measurements are shown in Table 3, with measurements taken across all shear ranges.

TABLE 3

| | | 700 nm/700 nm |
|---|---|---|
| | Smooth PUU | textured PUU |
| Mean Area (μm²) | 3.13 ± 0.19 | 3.46 ± 0.32 |
| Mean Perimeter (μm) | 6.39 ± 0.20 | 6.70 ± 0.32 |
| Mean Circularity | 1.08 ± 0.02 | 1.07 ± 0.03 |
| Index | | |

There was no substantial difference in the circularity index parameters between the two materials.

Adhesion and Thrombogenesis

The initial step in blood response to a synthetic surface is formation of an adsorbed protein coating, and unactivated platelets subsequently adhere to protein ligands adsorbed on the surface. Platelets then undergo activation and secretion, releasing granule contents, causing further aggregation of platelets into a plug that is strengthened by fibrin through the action of the coagulation cascade. Platelets play an active role in fibrin formation by providing the phospholipid membrane necessary for formation of the tenase and prothrombinase complexes. Removal of the platelet plug from the surface as an embolus leads to blockage of vessels downstream, leading to loss of oxygen and tissue death.

Hence, platelet adhesion to implanted blood-contacting biomaterials is a central event in surface-induced thrombogenesis. Once adhered, platelets can aggregate to form surface thrombi which may subsequently come off the surface as emboli.

The methods and apparatus described herein provide reduced surface adhesion of cells, other biological formed elements such as platelets, or other particulate materials to a surface. Such adhesion may be undesirable, for example, in medical implants such as blood vessel prostheses and blood pumps, or for objects upon which bacterial colonization is to be discouraged. Hence, surfaces designed according to the principles described herein may reduce the incidence of surface-induced thrombogenesis in blood-contacting devices and bacterial colonization in other systems.

Certain formed elements, such as platelets, change their behavior (are activated) through interaction with synthetic surfaces. Activation is generally undesirable, as it initiates downstream events such as, in the example of platelets, aggregation into clinically relevant emboli or potentiation of blood clotting. A synthetic surface having a sub-cellular topography may, through reduced access to the synthetic surface, provide a reduced opportunity for activation.

The platelet accessible surface area can be controlled by design of textures having features smaller than the platelet dimension, using common micro- and nano-fabrication techniques that do not require modification of the biomaterial surface chemistry. Reduced contact between platelets and potentially adhesive protein ligands on the material can reduce platelet adhesion forces, allowing physiological shear stresses to provide more effective surface washing when compared with smooth surfaces.

Properly selected nanoscale topographies on the surface of a biomaterial can lead to a reduction in platelet adhesion at physiological shear stresses. For example, a polyurethane surface can be patterned with arrays of pillars having dimensions and spacings ranging from 0.3 to 1.6 microns.

A platelet encountering a biomaterial that has been textured according to the principles described herein may exhibit the same or lower level of activation than does a platelet encountering to smooth polyurethane, and therefore reductions in platelet adhesion by nanotexturing may not be associated with greater activation of platelets in the bulk suspension.

Platelet activation can be determined for example by measuring expression of platelet activation markers, changes in adherent platelet morphology and activation of the complexes of the coagulation cascade. Activation can be studied by assessing platelet activation over time using dual-labeled flow cytometry. Surface topographies can thus be chosen to minimize platelet activation.

Most formed element-biomaterial adhesion phenomena are thought to be mediated by adhesion proteins. In the absence of such mediation the principle of reduced available surface area by selection of an appropriate sub-cellular topography is the same. Many synthetic materials are quickly coated by proteins upon contact with a biological system. Because the proteins are small compared with a properly selected sub-cellular topography, a protein coating that is conformal to a very rough approximation still results in the intended topography to be presented to the formed elements.

The protein coating may impose a minimum feature size on the chosen surface topography. A typical protein coating thickness may be around 20 nm, so that a ridge spacing of 40 nm would be expected to be filled by the protein coating. In such an example, the minimum feature dimension may be chosen as at least 3 times the protein coating thickness, or some other multiple of the protein coating thickness such as 4, 5, or 10 times the protein coating thickness. Feature sizes may also be chosen in anticipation of formation of a protein or other coat, for example by fabrication of 400 nm diameter posts when 500 nm diameter features are desired, in anticipation of formation of a 50 nm conformal coating.

One consideration may be the relative dimensions of the protein coating and the formed element for which reduced adhesion is desired. If the formed element has a dimension, such as a diameter, much greater than the protein coating, the topographic feature size can be chosen to be less than the diameter of the formed element, but much greater than that of the protein coating, for example at least 5 times greater, such as 10 times greater.

### Other Examples

Surfaces having desired surface topographies (such as described in this specification) may additionally be provided with surface coatings. For example, coatings such as anticoagulants and antiseptics may be provided to reduce initiation of blood clotting or discourage the growth of bacteria. Heparin or its derivatives can be provided as coating for the patterned surface. Such coatings, or the surface chemistry of the bulk material, may be chosen to either augment the effect of the surface texture by further discouraging formed element adhesion, or to complement the effect of the surface texture by diminishing or preventing the occurrence of undesirable phenomena other than formed element adhesion. For example, a blood-contacting surface may be provided with a subcellular texture as described herein to discourage platelet adhesion while also being provided with a bound heparin moiety to discourage biomaterial activation of the coagulation cascade.

Surfaces topographies may be provided having pillars, ridges, holes, grooves, and the like, or some combination of topographic features. For example, arrays of holes, each hole having a diameter less than the diameter of the formed element can be provided. As another example, ridges can be spaced less than the diameter of the formed elements.

Surface topographies may comprise rectangular pillars and ridges, or rounded structures such as sinusoidal profiles. Surface topographies may comprise features having two or more size scales, to reduce adhesion with two or more formed elements having different dimensions. Typically, however, a topography that discourages adhesion of one type of formed element is likely to discourage adhesion of moderately larger formed elements.

For any topography, the heights of the features can be larger than the reach of any adhesion proteins through which cells form attachments to surfaces. The widths of the features that contact the formed elements can be minimal, so as to provide as little accessible surface area as possible, but can be large enough that the features are self supporting and do not collapse under the influence of any fluid flow that might be present in a given application. Features may be provided with particular shapes to discourage deformation such as pillars having “X” or “C” shaped cross-sections or ridges having wavy shapes. The features that contact the formed elements can be spaced far apart so as to deprive the formed elements of available surface area, yet not be so far apart that formed elements can access the spaces between these features.

We demonstrated that 300 nm pillars are self-supporting when cast in a biomedical polyurethane. In preferred examples relating to platelet adhesion reduction, pillar width and pillar spacing are less than or approximately equal to 1 micron. Preferably, biomedical implants have a surface having topographic features that reduce the accessible area to platelets. For example, pillars may have a pillar width and pillar spacing both in the range 300 nm to 1.6 micron, and a pillar height between 100 nm and 1.6 micron. Platelets have an approximate diameter of 1 micron, so the spacing and width may both be less than 1 micron. In preferred examples, the topographic features are pillars having a pillar width of 300 nm to 700 nm, pillar spacing in the range of 400 nm to 700 nm, and pillar height in the range of 100 nm to 600 nm.

Vascular Grafts

Conventional smooth-walled vascular grafts and vascular grafts having supracellular textures are prone to thrombosis and clogging at diameters less than 5 mm, particularly at diameters less than 4 mm, and are generally seen as impractical for diameters less than 3 mm. By providing vascular grafts having an inside wall surface having topograph features such as described in this specification, improved vascular grafts can be provided. These improved grafts may have diameters less than 5 mm, even less than 3 mm, with a much reduced danger of failure.

A two-stage replication process can be used, such as described above. In one approach, a first surface (such as an etched silicon wafer, resist layer on silicon, metal or plastic sheet) is provided with the desired topography. A negative image is then formed on one surface of a flexible film negative, such as a silicone rubber film as described above. The flexible film can then be wrapped around the rod, providing the negative image of the desired topography on the outer surface.

The rod can then be coated with a polymer, so as to provide a polymer tube having the desired surface topography on the inner surface. For example, dip casting of the rod in polyurethane can be repeated until a desired wall thickness is obtained according to the desired mechanical properties of the finished graft. The rod can then be mechanically removed, e.g. by pulling it out of the polymer tube, and the flexible film negative may then be peeled from the inner wall. Other removal techniques may include mechanically, chemical, and/or thermal methods. For example, a liquid may be forced between the flexible film negative and the polymer, so that the polymer expands slightly and is separated from the negative. The textured polymer may form the entirety of the graft, or may be combined with an adventitial layer, again in accordance with the desired mechanical properties and the manner in which the designer intends the graft to integrate with surrounding tissues.

The flexible film can held in place upon the rod by any convenient method, for example by use of an adhesive or by use of a hollow rod having multiple small holes on its outer surface in order to permit retention of the film by vacuum.

Alternatively, vascular implants can be formed directly forming a tube from a polymer sheet having the desired surface topography. The seam can be sealed by adhesive, welding, or other method.

Alternatively, a rod can be provided having a negative image of the desired surface topography on the outer surface, and used as a master to mold the inside surface of the polymer tube. The rod shaped form can be formed by any desired method, such as laser ablation, stamping, or molding. The rod can be coated with polymer, for example by dip coating, and removed mechanically when the polymer tube is obtained.

Cardiac Valves

Polymeric cardiac valves are conventionally formed by casting on rigid forms. The anti-adhesive surface texture may be provided on the surface of a polymeric cardiac valve by covering the conventional rigid form with a flexible film negative as described above prior to casting of the valve. As described above, the flexible film negative may be held in place by use of adhesive, vacuum or the like. Alternatively, the rigid form may be provided with a negative of the desired pattern by laser ablation, stamping or molding.

Other Applications

Films having surface topographies according to the present invention can be adapted for use as diaphragms for circulatory support devices. Such diaphragms may need to be between 3 and 4 inches in size to fit into pulsatile or positive displacement devices that provide a volume on each beat similar to that of the adult natural heart.

Textured films nearly six inches in diameter can be created using conventional lithographic methods in conventional equipment as described herein. A flexible film, having a negative of the desired surface topography, can be shaped to any desired form, such as a curved surface, and polymer sheets cast or otherwise formed having the desired surface topography. For example, a silicone negative may be stretched slightly in order to conform to a mold or fixture having the desired shape and may be held in place by means of vacuum or a suitable adhesive. Alternatively, molds used to cast diaphragms, other chamber parts, complete chambers, or intermediate molds may be provided with the desired patterns by laser ablation, stamping, molding or the like.

Surfaces can also be provided having reduced bacterial adhesion, for example for use in medical implants (such as blood contacting implants), shunts, other medical applications, food packaging, surface cleanliness (such as food preparation devices, cookware, and food preparation surfaces), medical instruments, dental implant surfaces, orthopedic implants, selective (size-differentiated) bacterial culture, and the like. Other examples include reducing bacterial adhesion to any surface which may come in contact with a consumable item, such as beverage handling vats and pipes, grain handling equipment, and the like. Chemical coatings can also be provided, for example for enhanced sterilization, inhibition of bacterial slime formation, or other purpose. A surface topography can be chosen having e.g. a pillar spacing less than the relevant dimension (e.g. diameter) of a bacterium. Such surfaces can reduce initial colonization by bacterial films.

Surfaces according to examples of the present invention may be formed in polymers or other materials impregnated with, or otherwise releasing, an antibiotic, oxidizing agent, reducing agent, UV light, or having one or more other pathogen-resisting property. Other applications include forming low friction surfaces, and surfaces that resist liquid beading (if the feature size is less than a typical liquid bead size), for example including vehicle finishes and chemical engineering processing equipment surfaces.

Formed Elements

Formed elements, the adhesion of which to surfaces can be reduced, include blood cells (such as red blood cells, white blood cells), platelets, bacteria, or other particles. Formed elements have an effective dimension in relation to interaction with a surface. For example, for a spherical formed element, the effective dimension may be the diameter. For an ovoid, the effective dimension may be the major (larger) or smaller (minor) diameter, depending on how the ovoid interacts with the surface. For a rod, the effective dimension may be the length or diameter of the rod. Formed element dimensions may be the dimension of the formed element plus or minus a factor due to coating, oxidation, molecular interactions, or other process.

Formed elements can also include droplets of a liquid within another fluid (such as oil droplets in water and other emulsions, aerosol droplets, and the like), gas bubbles within a liquid, liquid-walled bubbles in a gas, viruses, prions, macromolecules (such as polymers, DNA, and the like), dust, particulate pollutants, other microorganisms, micellar structures, particles within sols, and the like.

Surfaces according to examples of the present invention may also be formed by adhesion of nanorods to surfaces, for example in a process including electrostatic deposition.

Feature Dimensions and Other Applications

The surface can be provided with a number of topographic features, such as a repeated pattern of pillars, holes, ridges, trenches, or the like. The surface can have a feature dimension, which may be pillar spacing, groove width, ridge spacing, other distance between repeated topographic features, or can correspond to the dimension of a feature itself, such as a width, thickness, or diameter of pillar, hole, ridge, or the like.

To encourage formed elements to interact with a surface within a certain portion of the surface, the surface other than that portion can be provided with topographic features such as those described herein.

Surfaces according to the present invention can be used in relation to applications where adhesion of formed elements is disadvantageous, such as medical or veterinary implants, devices in contact with blood, devices handling or in contact with other bodily fluids and tissues, surfaces which are desired to remain sterile, optical surfaces which are required to remain clean, inside walls of pipes, and the like.

Certain examples discussed above contemplate microscopic formed elements, where the formed element dimension may be less than 100 microns, or less than 10 microns. Here, the term microscopic also includes nanoscale formed elements, having a formed element dimension less than 1 micron. However, the principles described herein can also be applied to reduced adhesion of larger formed elements to a surface, such as grains, plant products, particles, and the like.

Surfaces according to the present invention can also be used in the cultivation and/or handling of formed elements, chemical engineering applications, food processing, fluid handling applications, and the like.

If the feature dimension is reduced in use, e.g. by a coating which forms on the surface, the feature dimension of a surface before use can be greater than the formed element dimension.

For reduced adhesion of a formed element to a surface having a feature dimension, the feature dimension can be chosen to be less than the dimension of the formed element. For example, for an array of rectangular pillars, the pillar spacing may be defined as the distance between the outer edges of neighboring pillars, and can be less than the formed element dimension. For rounded features, such as rounded pillars, the feature dimension may be defined as the spacing between the centers of the pillars, or between the outer edges at some position on the pillar relative to the top or base of the pillar.

For sinusoidal features or other geometric periodic features such as triangular features, the feature dimension may be defined as the repeat distance of the periodic feature.

Surfaces

Surfaces which may be provided with topographic features such as those described above include plastic, metal, semiconductors, glass, other dielectrics, and the like. Surfaces may be generally flat, the term ‘generally’ indicating distance scales greater than the feature dimension. Surfaces may also be generally curved (for example, the inner surface of a tube or conduit), rigid, or flexible. The manufacture of such topographic features may include steps such as etching, scoring, laser ablation, stamping, molding, adhesion of features to preexisting surfaces, self-assembly processes, and the like.

Topographic features may be arranged in periodic arrays, or in a random distribution where the feature dimension may be a statistical average. Topographic features may also have orientational alignment along a direction within the surface or at an angle to the (average) surface normal, for example in relation to a fluid flow direction relative to the surface and/or desired or natural orientations of formed elements relative to the surface.

The feature dimension may be adjusted dynamically, for example by compression, stretching, curving, thermal methods, actuation of possibly microscale elements, electrostriction, or other process or processes. For example, a silicone rubber or other elastic negative may be stretched, compressed, or otherwise distorted before casting of a synthetic polymer thereon, and feature distribution in the original pattern may be determined in anticipation of said distortion.

Hence, an improved method of manufacturing a vascular graft, prosthetic valve or blood pump chamber from a polymer is provided, wherein a flexible polymer sheet having a negative pattern is fixed to a generally cylindrical rod, and an interior surface of the vascular graft is formed by casting a polymer upon the negative pattern. Patents or publications mentioned in this specification are herein incorporated by reference. Methods, compounds, and apparatus described herein are exemplary, and are not intended as limitations on the scope of the invention. Changes therein, different combinations of described elements, alternatives, and other applications and approaches will occur to those skilled in the art, which are encompassed within the spirit of the invention as defined by the scope of the claims. U.S. Provisional Patent Application Ser. No. 60/561,350, filed Apr. 12, 2004, is incorporated herein by reference.

1. A surface providing reduced adhesion to formed elements, the formed element having an element dimension,
the surface having an accessible area over which the formed elements can adhere to the surface,
the surface having a plurality of topographic features, the topographic features having a feature dimension less than the dimension of the formed element so as to reduce the accessible area of the surface.

2. The surface of claim 1, wherein the topographic features include protrusions, the feature dimension being a protrusion spacing between the protrusions.

3. The surface of claim 2, wherein the protrusions have a protrusion width, the protrusion width being less than the dimension of the formed element.

4. The surface of claim 2, wherein the protrusions are pillars, the feature dimension being a pillar spacing between the pillars.

5. The surface of claim 4, wherein the pillar spacing is between 300 nanometers and 1 micron.

6. The surface of claim 4, wherein the pillars each have a pillar width and a pillar height, the pillar width being between approximately 100 nm and 1000 nm, and the pillar height being between approximately 100 nm and 1000 nm.

7. The surface of claim 6, wherein the pillar width is a diameter of an approximately circular pillar or an edge length of an approximately rectangular pillar.

8. The surface of claim 5, wherein the formed elements are platelets, the surface providing reduced adhesion to platelets.

9. The surface of claim 1, wherein the topographic features include indentations, the feature dimension being a width of the indentation.

10. The surface of claim 1, the surface being formed in a polymer.

11. The surface of claim 10, wherein the polymer is part of a blood pump or vascular implant exposed to a biological fluid including the formed elements.

12. The surface of claim 10, wherein the polymer is a polyurethane.

13. The surface of claim 10, wherein the polyurethane is a poly(urethane urea).

14. An apparatus having a surface exposed to formed elements, the surface having a reduced adhesion to the formed elements,
the surface having topographic features having a feature size less than the dimension of the formed element so as to prevent the formed element from accessing a portion of the surface.

15. The apparatus of claim 14, wherein the apparatus is intended for implantation into a human or non-human animal so that the surface contacts blood, the formed elements being platelets, the dimension of the formed elements being a platelet diameter.

16. The apparatus of claim 14, wherein the topographic features comprise an array of pillars.

17. The apparatus of claim 16, the pillars having a pillar width between 300 nm and 1 micron, and a pillar spacing between 300 nm and 1 micron.

18. The apparatus of claim 14, wherein the surface is a polymer surface.

19. The apparatus of claim 18, wherein then polymer is a polyurethane.

20. The apparatus of claim 14, wherein the apparatus is a vascular graft comprising a polymer tube, the surface being an interior surface of the polymer tube.

21. A method of fabricating a biomedical implant having at a surface having topographic features providing reduced platelet adhesion, the surface being a surface of a polymer, the method comprising:
providing a master surface having a representation of the topographic features;
fabricating a negative having a negative representation of the mask surface;
fabricating a polymer upon the negative, the polymer providing the surface having the topographic features;
fabricating the biomedical implant using the polymer, the surface having topographic features that reduce platelet adhesion to the biomedical implant.

22. The method of claim 21, wherein the polymer is a polyurethane.

23. The method of claim 21, wherein the flexible negative comprises a silicone rubber.

24. The method of claim 21, wherein the polymer is a polymeric biomaterial.

25. The method of claim 21, wherein the topographic features are pillars.
